(19) United States
(12) Patent Application Publication
(10) Pub. No.: US 2024/0358999 A1
(43) Pub. Date: Oct. 31, 2024

(54) BLOOD PUMP WITH MAGNETICALLY SUPPORTED ROTOR

(71) Applicant: Berlin Heart GmbH, Berlin (DE)

(72) Inventors: Hendryk Richert, Berlin (DE); Oliver Peters, Berlin (DE)

(73) Assignee: Berlin Heart GmbH, Berlin (DE)

(21) Appl. No.: 18/444,245

(22) Filed: Feb. 16, 2024

Related U.S. Application Data
(63) Continuation of application No. PCT/EP2022/073356, filed on Aug. 22, 2022.

(30) Foreign Application Priority Data
Aug. 20, 2021 (EP) 21192477.4

(51) Int. Cl. A61M 60/422; A61M 60/10; A61M 60/216; A61M 60/82

(52) U.S. Cl. CPC A61M 60/422; A61M 60/10; A61M 60/216; A61M 60/82

(57) ABSTRACT

A pump, such as a blood pump, includes a pump chamber with a wall and a fluid connection between an inlet and an outlet, along with an impeller, a motor stator, and a control coil arranged on the pump chamber for exerting, together with first rotor magnets, a controllable axial force on the rotor. A control unit provides a control current for the control coil, in order to hold the rotor in a position which is spaced apart axially from the wall of the pump chamber. A first chamber magnet is arranged close to the winding of the control coil and opposite the first rotor magnet, in order to provide, together with the first rotor magnet, a force between the first rotor magnet and the first chamber magnet, and in order to orient the rotor relative to the wall of the pump chamber.

### PRIORITY

This application claims priority as a Continuation of PCT/EP2022/073356, filed on Aug. 22, 2022, entitled “PUMP”, published as WO 2023/021222 A1, which claims priority to European Patent Office Application No. EP 21192477.4 filed on Aug. 20, 2021. The entire disclosure of the above applications are incorporated herein by reference.

### TECHNICAL FIELD

The application relates to a pump, in particular a pump with a magnetically supported rotor, such as a blood pump, with a magnetically supported rotor, a method for operating the pump and use of the pump. The pump can be used for example in a cardiac support system.

### BACKGROUND

Cardiac support systems are used to maintain blood flow in the body, in particular in cases where a heart cannot guarantee this sufficiently.

Blood pumps with a magnetically mounted rotor, in brief magnetically mounted blood pumps, are currently regarded as the prior art in the field of blood pumps which can be used in cardiac support systems for example. However, the complexity of a structure comprising magnetic bearings can prevent a miniaturization of the blood pump.

### SUMMARY

The present invention is based on the problem of enabling a further miniaturization of a pump and in particular a magnetically mounted blood pump, at the same time as maintaining important functional properties such as performance, controllability, safety and hemocompatibility. The problem is solved according to the features of the independent claims. Further advantageous embodiments of the invention are given in the claims which refer back to independent claims.

A pump is disclosed that comprises a pump chamber with a wall and a fluid connection for a flow of fluid between an inlet and an outlet of the pump chamber. The pump can be for example, but also in particular, a blood pump and the fluid flow can be for example, but also in particular, a blood flow. The fluid flow is achieved in that the fluid flows through the inlet, which is an opening of the pump chamber, into the pump chamber, passes through the pump chamber and leaves the pump chamber through the outlet, which is an opening of the pump chamber. The wall delimits the pump chamber, which is in the form of a hollow chamber. The pump has the task of driving the fluid flow.

The pump further comprises an impeller arranged in the pump chamber and connected to the rotor, configured for rotating about an axis of rotation, which is aligned with the inlet and defines an axial direction. The impeller is configured to drive the fluid flow from the inlet to the outlet. For example, the impeller can be rigidly connected to the rotor. The rotor and/or the impeller can be aligned for example such that the axis of rotation runs approximately through the center of the inlet. It is assumed that the rotor only performs small translational movements so that, with the aim of a simpler and more comprehensible description, the axis of rotation can be regarded here as fixed with respect to the pump chamber. The impeller can have one or more sections, for example blades and/or vanes, with which the fluid, for example blood, can be moved, with which the fluid flow can be driven. For example, the impeller can have a substantially disc-like form, wherein this also includes in particular a closely fitting envelope around the impeller, into which the blades or vanes can be integrated for example. A disc-like form is for example a substantially cylindrical shape, wherein the cylinder height is lower than the diameter of the cylinder base area, for example by a factor of 2 to 3.

In addition, the pump comprises a motor stator arranged at the pump chamber, wherein the motor stator comprises at least one motor coil, which is configured to provide a motor magnetic field, which is configured to interact with a magnetic field of a motor magnet arranged at the impeller in order to rotationally drive the impeller. It is possible here that the motor stator comprise a plurality of motor coils. These can be arranged in a ring for example. Windings of the coils can for example each comprise turns about the axial direction. The pump chamber and motor stator can be separated from one another by the wall.

It is also possible that the motor magnet comprises a plurality of motor magnets. The motor magnets can be arranged in a ring at the impeller for example. It is also conceivable that the motor magnets are positioned opposite the motor coils. The magnetic field direction of the motor magnet(s) can be directed in an axial direction. In the case of adjacent motor magnets it is also possible for the magnetic field direction to deviate by 180°, wherein other deviations are also possible, such as for example 90°. The motor stator and motor magnets can interact, for example according to the principle of an axial flow motor.

In addition, the pump has a control coil arranged at the pump chamber, wherein one or more turns of a winding of the control coil are arranged at the wall around the pump chamber and the control coil is configured to generate a magnetic field by means of a control current, which magnetic is configured to exert a controllable axial force at the rotor in the axial direction with a first rotor magnet arranged at the rotor.

It is possible for the control coil to be arranged near the inlet. For example, it can be arranged in such a way that its winding is arranged, for example completely, when viewed in axial direction, between the inlet and rotor. On the other hand, it is also possible to have a different arrangement, for example so that the control coil is located near the impeller. In each case, it is conceivable that the control coil is not located in the pump chamber, but is positioned behind the wall starting from the pump chamber. The control coil can also be integrated into the wall. The one or more turns of the winding of the coil can be guided about an axial direction. It is also possible that the controllable axial force acts in the direction of the inlet or away from the inlet.

For example, the first rotor magnet can be permanently magnetic and ring-shaped and can be guided around a circumference of the rotor, for example an inner circumference and/or an outer circumference, around the rotor, for example so that the first rotor magnet has a substantially constant distance from the axis of rotation in radial direction along the circumference. The first rotor magnet can then for example have a magnetization and thus a magnetic field direction in the radial direction. Magnetization includes, inter alia, the arrangement of the north and south poles of a magnet in relation to one another. A magnetization direction indicates a direction running in a straight line from the north pole to the south pole of the magnet. In the following, the term magnetization can also be used within the meaning of a direction of magnetization, particularly when referring to directions.

However, the first rotor magnet can also have a magnetization in axial direction and it can also be divided into one or more sections, for example in the axial direction or also along its circumference, each with its own magnetization. The magnetization direction of the various sections can differ from one another, for example in opposite directions. For example, an arrangement in the form of Halbach array is also conceivable, wherein in such a case the rotor magnet can be divided into sections, whose direction of magnetization corresponds to the specifications of a Halbach array. The rotor magnet can be configured for example as a neodymium magnet, but other magnet materials are also possible.

The pump also has a control unit which is configured to provide the control current for the control coil in order to maintain the rotor in a position spaced apart axially from the wall of the pump chamber. The control unit can be, for example, a microcontroller, a digital signal processor, an FPGA (field programmable gate array) or an ASIC (application specific integrated circuit) or also a combination thereof. Furthermore, additional electronic components and corresponding circuits can be added to the control unit, for example one or more sensors, such as for example a distance and/or position sensor, a magnetic field sensor, a speed sensor, a pressure sensor, a sensor for voltage measurement and/or current measurement, a flow sensor for the fluid and the like, but also for example an amplifier, a filter, an inverter or also a circuit, which is used for communication, a voltage supply etc.

The pump also has a first chamber magnet, which is arranged at the pump chamber, close to the winding of the control coil and opposite the first rotor magnet, and is configured, together with the first rotor magnet, to provide a force between the first rotor magnet and the first chamber magnet in order to align the rotor relative to the wall of the pump chamber. Close to the winding means here that the first chamber magnet can be positioned for example adjacent to the winding of the control coil or only at a small distance, at which the magnetic field of the first rotor magnet arranged opposite the chamber magnet can interact with the magnetic field of the control coil.

In one embodiment the control coil is arranged upstream of the first chamber magnet. In a further embodiment, the control coil is arranged both upstream and downstream of the first chamber magnet. In a further embodiment a first and a second control coil are provided, wherein the first control coil is arranged upstream and the second control coil is arranged downstream of the first chamber magnet. The downstream portion of the control coil or the second control coil can be arranged upstream of a second chamber magnet. The first chamber magnet can have a ring shape for example. It is conceivable that the first chamber magnet is integrated into the wall of the pump chamber, for example also in a projection of the wall of the pump chamber, or is also positioned behind or in front of the wall starting from the pump chamber. The first chamber magnet can for example have a substantially constant distance from the axis of rotation along a circumference. The first chamber magnet can also extend annularly about the axis of rotation. It is also possible that the first chamber magnet for example has a magnetization in the radial direction. However, it can also have a magnetization in the axial direction and it can also be divided into one or more sections, for example in the axial direction or also along its circumference, each with its own magnetization. The magnetization direction of the various sections can differ from one another, for example in opposite directions. However, for example an arrangement in the form of a Halbach array is also possible, wherein in such a case the first chamber magnet can be divided into sections, whose direction of magnetization corresponds to the specifications for a Halbach array. The first chamber magnet can be configured for example as a neodymium magnet, but other magnet materials are also conceivable.

The pump can also have a second magnetic bearing, wherein the second magnetic bearing can for example comprise a second chamber magnet, which is arranged at the pump chamber where the axis of rotation meets the wall. The second magnetic bearing can also have a second rotor magnet arranged opposite the second chamber magnet at the rotor. It is possible that the second magnetic bearing provides a repelling force between the second rotor magnet and the second chamber magnet, in order to maintain the rotor or a section of the rotor in a position spaced apart from the wall of the pump chamber in a radial direction defined perpendicular to the axial direction. The second magnetic bearing can function for example according to a principle of a permanent magnetic bearing or a passive magnetic bearing.

The second chamber magnet can for example have a cylindrical or also an annular form. The second chamber magnet is arranged at the pump chamber. It is conceivable here that the second chamber magnet is integrated into the wall of the pump chamber, for example also into or onto a projection of the wall of the pump chamber, or is also positioned behind or in front of the wall, starting from the pump chamber. The second chamber magnet can for example have a substantially constant distance from the axis of rotation along a circumference. The axis of rotation can also run through the second chamber magnet and the second chamber magnet can also extend annularly about the axis of rotation. It is also possible for the second chamber magnet to have magnetization in the radial direction for example. However, it can also have a magnetization in the axial direction and it can be divided into one or more sections, for example in the axial direction or also along its circumference, each with its own magnetization. The magnetization direction of the various sections can differ from one another, for example in opposite directions. However, it is also conceivable to have an arrangement in the form of Halbach array, wherein in such a case the second chamber magnet can be divided into sections, whose direction of magnetization corresponds to the specifications for a Halbach array. The second chamber magnet can be configured as a neodymium magnet for example, but other magnet materials are also conceivable.

In one variant it is provided that the second rotor magnet is configured to be permanently magnetic and ring-shaped, and is guided around the rotor on a circumference of the rotor, for example on an inner circumference and/or an outer circumference, for example such that the second rotor magnet has a distance from the axis of rotation that is substantially constant in the radial direction around the circumference. The second rotor magnet can then for example have a magnetization in the radial direction. However, it can also have a magnetization in axial direction and it can also be divided into one or more sections, for example in the axial direction or also along its circumference, each with its own magnetization. The magnetization direction of the various sections can differ from one another, for example in opposite directions. For example, an arrangement in the form of Halbach array is also conceivable, wherein in such a case the rotor magnet can be divided into sections, whose direction of magnetization corresponds to the specifications of a Halbach array. The rotor magnet can be configured for example as a neodymium magnet, but other magnet materials are also possible.

In some embodiments it is possible for the first rotor magnet and the second rotor magnet to be identical. This can be advantageous for example, where it is important to reduce the number of components required to manufacture the pump and/or reduce the number of manufacturing steps. This option is suitable for example if the magnetization of the first and second rotor magnet is meant to be identical and both are to be arranged adjacent to one another.

In some embodiments, it is conceivable that the first rotor magnet and the second rotor magnet are different from one another. This can be advantageous for example, if it is important to adjust the forces provided by the respective rotor magnet, for example in cooperation with the respective associated chamber magnet, as described above, or for example the control coil, separately at the rotor, because this can be easier for example, or to adjust the respective force application point at the rotor separately and thus in a better way for example.

In one exemplary embodiment it is possible that the rotor has a recess around the axis of rotation, and is configured to guide the fluid flow through the recess. The rotor can be formed for example in one section as a hollow tube or also as a bore. It is possible that the fluid flow, for example the blood flow, can flow through the recess. Thus in one example this also makes it possible to minimize the distance between the first rotor magnet and the first chamber magnet, without also having to minimize the fluid flow through the pump. As the force effect between the first rotor magnet and the first chamber magnet increases with a smaller distance from one another, the magnets can be configured to be smaller and/or less powerful at a smaller distance. This makes it possible for example, to reduce a volume of the pump and/or reduce the manufacturing costs while maintaining the same performance parameters.

It is conceivable that the force between the first rotor magnet and the first chamber magnet is a force of attraction and the first rotor magnet and the first chamber magnet form a first magnetic bearing, which is configured to orient the rotor in a tilting direction, which has an axis of rotation in the radial direction. For example it is possible for the first rotor magnet and the first chamber magnet to be arranged in the vicinity of an outer circumference of the impeller. Thus the force of attraction between the first rotor magnet and first chamber magnet can ensure that the impeller is always positioned on its outer circumference in the vicinity of the chamber magnet, i.e. that it does not rotate in tilting direction, which can also be referred to as tilting. The impeller can thus be held in a favorable position for driving the fluid flow.

In one exemplary embodiment it is also possible that the force between the first rotor magnet and the first chamber magnet is a repelling force in a radial direction, defined perpendicular to the axial direction, and the first rotor magnet and the first chamber magnet form a first magnetic bearing, which is configured to maintain the rotor in a position spaced apart from the wall of the pump chamber in the radial direction. The first magnetic bearing can function for example according to the principle of a permanent magnetic bearing or a passive magnetic bearing. The first magnetic bearing can in this case be a magnetic bearing which is stable in radial direction. In this way it is also possible for example to support the rotor completely in the radial direction in cooperation with the second magnetic bearing. When the first and second magnetic bearing are spaced apart from one another, for example in the axial direction, a movement of the rotor in the tilting direction can also be suppressed or at least reduced. This can be achieved for example in that the first magnetic bearing is arranged close to the inlet in axial direction and the second magnetic bearing is arranged close to the outlet, for example also close to the impeller. In such a case, the rotor, viewed in axial direction, can have a first section close to the inlet and a second section remote from the inlet. In one example it is conceivable that the first magnetic bearing is arranged in the first section and the second magnetic bearing is arranged in the second section. The impeller can then also be arranged in the second section for example. The first and second sections can merge seamlessly into one another.

In a further embodiment the first magnetic bearing is configured alternatively or additionally to the repelling force described above in radial direction such that the force between the first rotor magnet and the first chamber magnet is a repelling force in axial direction, and the first rotor magnet and the first chamber magnet form a first magnetic bearing, which is configured to hold the rotor in a position spaced apart from the wall of the pump chamber in axial direction. Furthermore, the second magnetic bearing can also be configured such that it supports the action of the first magnetic bearing.

In addition, it is provided in one embodiment that the control coil has a magnetically conductive material arranged between the winding and the fluid connection and/or on a side of the control coil facing away from the rotor. A magnetically conductive material can have a high relative magnetic permeability, for example such as a ferromagnetic material, for example iron. Typical values for the relative magnetic permeability of ferromagnetic materials can for example lie in the range between 300 and 10000 (without unit), wherein for certain materials, such as for example mu-metals, amorphous metals or also nanocrystalline metals are known with values significantly higher, for example up to 150000 or even up to 500000. Lower relative magnetic permeabilities are also known for certain materials, which can drop to around 4 with ferrites for example. The magnetically conductive material can be used to improve the conduction of a magnetic flow provided by the control coil and to guide the magnetic flow, so that the magnetic field generated by the control coil can generate a greater axial force when interacting with the magnetic field of the first rotor magnet. The magnetic flow is guided in such a way that magnetically conductive material can be provided where there is no force effect between the magnetic field of the control coil and the magnetic field of the first rotor magnet. Accordingly, the magnetically conductive material can be arranged on a side of the control coil facing away from the rotor, for example on a side of the control coil radially remote from the axis of rotation or also on a side of the control coil axially facing the inlet. Furthermore, it is possible to arrange the magnetically conductive material on a side of the control coil radially facing the axis of rotation, if no axial force is to be generated from there in the interaction between the control coil and first rotor magnet. This may be the case for example, if the winding of the control coil is arranged fully between the inlet and an end of the rotor facing the inlet or, viewed in the axial direction, is arranged between the first rotor magnet and the impeller.

However, other, alternative and/or additional options for an arrangement are also possible. For example, the control coil can consist of magnetically conductive material which is arranged on a side of the control coil facing away from the fluid connection and/or on a side of the control coil facing the rotor. The side of the control coil facing away from the fluid connection can also be the side of the control coil radially remote from the axis of rotation or partially coincide therewith. It is also conceivable that a magnetic force effect with the first rotor magnet should not be generated from all areas of the control coil facing the rotor. The magnetically conductive material can therefore also be arranged in these areas. As significant portions of the magnetic flow generated by the control coil are localized in the magnetic conductive material, substantially no force effect is to be expected between the control coil and the first rotor magnet starting from the areas in which the magnetically conductive material is arranged. In order to achieve a force effect between the control coil and first rotor magnet, it can be advantageous not to arrange any magnetically conductive material in at least one area of the control coil.

The advantages of the arrangement of the magnetically conductive material are at least twofold: on the one hand, the arrangement of the magnetically conductive material can increase the effect of force between the control coil and the first rotor magnet while the control current remains constant. Furthermore, in this way also a location of the force effect can be determined. The pump can thus achieve greater efficiency. For example, this can also mean that the pump can be configured to be smaller while having the same power.

In one embodiment the pump can comprise a biasing magnet arranged at the control coil, which is configured to bias the magnetic field of the control coil. The biasing magnet can be a permanent magnet for example. In this connection biasing means for example that a permanent magnetic field is provided by the biasing magnet, which can be superimposed on the magnetic field of the coil. It is possible for example that the biasing magnet is at least partly adjacent to the magnetically conductive material, which is arranged at the control coil and/or at the control coil itself. In this way, the biasing magnet can be integrated into a magnetic circuit, which includes the control coil and optionally the magnetically conductive material. The biasing magnet can for example cause a magnetic flow in the magnetic circuit. The magnetic flow generated by the control coil can then be superimposed on the magnetic flow of the biasing magnet in the magnetic circuit. By integrating the biasing magnet into the magnetic circuit of the control coil it is possible for example to create a force effect between (the magnetic circuit of the) control coil and the (magnetic field of the) first rotor magnet, even without a control current. By applying a control current to the control coil this force effect can be increased or decreased, i.e. controlled, depending on the amount and sign of the control current. For example, the biasing magnet can be used to set a force-related operating point of the pump in the axial direction which improves the controllability of the pump.

Another way of biasing the magnetic field of the control coil is for example to use another coil as a biasing magnet or even the control coil itself, in which a direct current is fed into the coil or the control coil. However, in contrast to a permanent magnet as a biasing magnet, this may require an electric current.

The pump can be configured so that the motor magnet is arranged on a side of the impeller facing away from the inlet, for example if the motor stator is also arranged on a side of the impeller facing away from the inlet. The motor magnet can then be arranged so that there is a small distance between the motor magnet and the motor stator. As the force effect between the magnetic field of the motor stator and the magnetic field of the motor magnet increases with decreasing distance, the motor magnet and motor stator can be configured to be smaller overall while maintaining the same force effect. This allows the available installation space to be used efficiently and the controllability of the pump to be improved.

On the other hand it is also conceivable that the motor magnet is arranged on a side of the impeller facing the inlet. This can be advantageous for example, if for example the motor stator is also arranged on a side of the impeller facing the inlet. The motor magnet can then be arranged so that there is a small distance between the motor magnet and the motor stator. As explained above, the motor magnet and motor stator can thus be configured to be smaller overall while maintaining the same force effect.

In one exemplary embodiment it is possible that the control unit is configured to provide a motor coil current such that the motor magnetic field, together with the magnetic field of the motor magnet, creates a force in the axial direction between the motor magnet and the motor coil. For example, a part of a field-orientated control of the motor coil current, a current can be calculated and controlled or regulated, such that not only a rotary driving force acts on the rotor via the magnetic field of the motor stator and the magnetic field of the motor magnet, but also an axially acting force is created on the rotor by the motor coil current. The axially acting force can be controlled for example such that it increases or decreases depending on the speed of the rotor. It can also be controlled such that it increases or decreases according to a position of the rotor, for example a position in axial direction. The axially acting force can also be controlled such that the rotor can be held in a preferred axial position, for example in an axial position at a predetermined distance from the wall or also in an axial position, in which the power consumption of the pump is low. The latter is the case for example for an axial position in which all axial forces on the rotor, which cannot be attributed to the motor coil current and/or the control current of the control coil add up to zero. For this purpose for example, a suitable method can be implemented in the control system, for example a control method or a combination thereof, for example a zero force control. The method implemented in the control system can for example process a signal provided by one of the sensors, such as a position signal, a magnet signal, a pressure signal, a fluid flow signal, a current signal, a voltage signal and/or also a speed signal.

Both the first and the second magnetic bearing can each have a radial rigidity, i.e. rigidity in the radial direction, wherein the rigidity indicates a ratio of an acting force to an associated displacement path length. In this case, the radial rigidity indicates an external force acting on the magnetic bearing in the radial direction, which is required for displacing the rotor on the magnetic bearing by a specified path length in radial direction. The radial rigidity is a property of the respective magnetic bearing, which can depend for example on the respective distance between the respective rotor magnet and the respective chamber magnet and/or for example on the magnetic material used and/or the respective magnetization and/or the direction of magnetization. The possible arrangement as a Halbach array can also affect the respective rigidity of the respective magnetic bearing. In general, the rigidity of a magnetic bearing can depend on the magnetic field strength between the respective magnets.

In one exemplary embodiment it is possible that a ratio of the radial rigidity of the second magnetic bearing to the radial rigidity of the first magnetic bearing is between 4:1 and 1.5:1 such as between 3:1 and 1.5:1. The respective rigidities are considered here. For example, the first magnetic bearing can have a lower rigidity than the second magnetic bearing. For example, it is possible that a radial force acting on the rotor, which can result for example from a flow and an acceleration of the fluid, is distributed to both magnetic bearings (first and second magnetic bearing) such that the rotor experiences no or only a small tilting moment, which can lead to a tilting of the rotor and thus a possible tilting of the rotor is suppressed or at least reduced. Here it is significant that a product of radial force (radial bearing rigidity multiplied by a deflection of the rotor in the radial direction) and lever arm between the force application point and the respective magnetic bearing center point (viewed in axial direction) is essentially the same for both magnetic bearings and thus the deflection in the radial direction at both magnetic bearings is also the same.

A pump is also conceivable in which the second rotor magnet and the second chamber magnet are arranged relative to one another so that an axial force acts on the rotor in axial direction away from the inlet. For example, the second rotor magnet and the second chamber magnet can be arranged offset to one another in axial direction.

Furthermore, it is also possible that a quotient of the radial rigidity of the second magnetic bearing to the radial rigidity of the first magnetic bearing remains essentially constant during a rotor displacement in the axial direction, in the radial direction or in the tilting direction, which has an axis of rotation in the radial direction, wherein in this example the radial rigidity of the first magnetic bearing can be composed of the rigidity of the control coil and the first chamber magnet with the first rotor magnet. This also means that the magnetic field of the control coil can influence the rigidity of the first magnetic bearing in addition to the magnetic field of the first chamber magnet.

In an arrangement of the motor, which comprises the motor stator with the motor coil and the motor magnets, and the control coil it is possible to configure an axial force generation in such a way that the sum of the forces generated by the motor and the control coil is substantially constant in the axial direction.

It is conceivable here for example that the force in the axial direction, which is generated by the motor, which may be an axial flow motor for example, is greater when the rotor, viewed in axial direction, is further away from the inlet than when the rotor, viewed in the axial direction, is closer to the inlet. The force generated by the motor in the axial direction can have a substantially linear or approximately linear dependence on the position of the rotor viewed in axial direction. It is also possible that the linearity or approximate linearity only applies in a spatially limited section, in which the rotor is located, whereby there may be several such section in which for example a different linear relationship may also apply in each case. In general, a non-linear relationship between axial rotor position and maximum force on the rotor is also conceivable.

It is for example also conceivable, that the force in the axial direction generated by the control coil is greater when the rotor, as viewed in axial direction, is closer to the inlet than when the rotor, viewed in axial direction, is further away from the inlet. The force generated by the control coil in the axial direction can have a substantially linear dependence on the position of the rotor viewed in the axial direction.

The pump can be used for example in a cardiac support system, for example as a blood pump or as a magnetically mounted blood pump.

A method for operating a pump is also disclosed. The method can also involve operating a blood pump or a magnetically mounted blood pump. For example, the method can be carried out by using a pump as described above. The features described in relation to the pump can also be applied to the method insofar as the features of the method can also be applied to the pump.

The disclosed method for operating a pump comprises the steps:
providing a motor magnetic field for driving an impeller connected to a rotor to drive a fluid flow from an inlet to an outlet of a pump chamber;
providing, with a control coil and a first rotor magnet arranged at the rotor, a controllable axial force on the rotor in the direction of an axis of rotation of the rotor, which defines an axial direction;
providing a control current for the control coil, to maintain the rotor in a position axially spaced from a wall of the pump chamber, such as in a time-varying position in which the sum of all forces on the rotor except the controllable axial force in the axial direction adds up to zero;
providing a force between the first rotor magnet and a first chamber arranged at the pump chamber near a winding of the control coil.

The said steps can be carried out in this order but can also be carried out in a different order. Individual or several of the steps can also be performed multiple times.

As part of the method a zero force control can be used in which a particularly small control current is required, as the rotor is held in the position which may vary over time in which the sum of all forces on the rotor except the controllable axial force in the axial direction add up to zero or essentially add up to zero. This enables an efficient operation of the pump with minimized electrical control power.

In one exemplary embodiment, the method for operating a pump may also comprise one or more of the following steps:
guiding the fluid flow through a recess in the rotor around the axis of rotation of the rotor away from the inlet towards the outlet;
guiding the fluid flow, so that the fluid flow at the inlet is guided substantially in the axial direction and at the outlet is guided in a direction that has substantially no axial component.
guiding a secondary fluid flow between the first rotor magnet and the wall of the pump chamber and/or guiding a tertiary fluid flow between the motor magnet and the wall of the pump chamber.

The guiding of the secondary and the tertiary fluid flow can be used for example for flushing the pump chamber. For example, in the case of a blood pump this can reduce the risk of thrombosis.

The method for operating a pump can also include the following step: controlling an electric current of the control coil such that an average power consumption of the control coil is minimized.

It is also possible that the method for operating a pump comprises the following step: generating a repelling force between a second rotor magnet and a second chamber magnet arranged at the pump chamber, where the axis of rotation meets a wall of the pump chamber.

The pump can thus also be used in many other technical areas than those mentioned above, in which a fluid flow must be driven, whereby fluids can be gases or liquids for example. In the context of this disclosure a preferred use is as a magnetically mounted blood pump, for example in a cardiac support system.

In the following, exemplary embodiments are shown with reference to the figures and explained in the following. In the Figures:

### BRIEF DESCRIPTION OF THE DRAWINGS

FIG. 1A shows a schematic representation of a pump;

FIG. 1B shows a schematic representation of a rotor of the pump from FIG. 1A;

FIG. 2A shows a schematic representation of a pump;

FIG. 2B shows further details of the pump of FIG. 2A;

FIG. 3A-3G show schematic representations of a first section of the rotor and a control coil in a partial cross-sectional and partially perspective view;

FIG. 4 is a diagram showing an option for generating axial force as a function of an axial position of the rotor; and

FIG. 5 is a flow chart for a method for operating a pump.

### DETAILED DESCRIPTION

Recurring elements in the Figures are denoted by the same reference signs and are partly omitted, particularly if no reference is made to these elements in relation to a specific drawing. It is also understood that the shown exemplary embodiments only represent options for implementing the disclosed inventive ideas and are not limiting in any way. All of the drawings are simply schematic or diagrammatic representations, even if this is not always explicitly indicated. Details which are not mentioned in the explanation may be omitted from the individual representations.

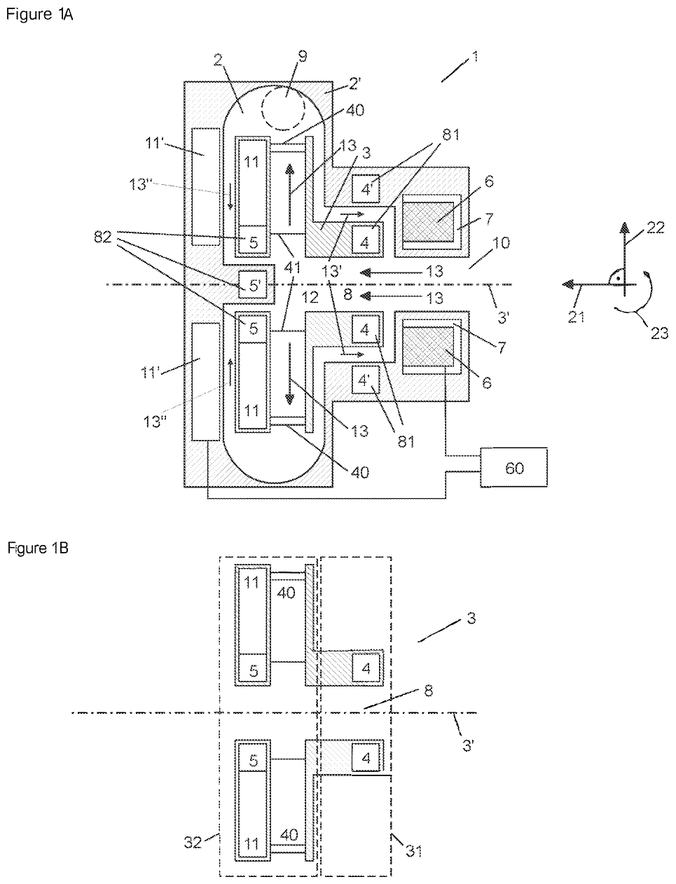

FIG. 1A shows a schematic representation of a pump 1, in particular a pump 1 with a magnetically mounted rotor 3. The pump may be a blood pump 1 and includes a magnetically mounted rotor 3. The representation in FIG. 1A is a cross-sectional view. The pump 1 has a pump chamber 2 which is delimited by a wall 2′. The material from which the wall 2′ is constructed can be metal, for example steel, in particular also stainless steel, or also titanium, for example also a titanium alloy or also a ceramic. In some applications however, the material of the wall 2′ can also be for example a plastic or for example an alloy or a combination of some or more of the aforementioned materials. The wall 2′ can be manufactured as multiple parts for example and assembled during the manufacture of the pump 1. The pump chamber 2 has an inlet 10 and an outlet 9 and a fluid connection 12, i.e. a volume in which fluid can flow, between the inlet 10 and the outlet 9. The inlet 10 is an opening in the wall 2′ which is for supplying fluid into the pump chamber 2 and the outlet 9 is an opening in the wall 2′ for discharging fluid from the pump chamber 2. For example, the fluid can be liquids or gases. In particular the fluid can be blood.

The pump 1 in FIG. 1A also comprises a rotor 3 which is configured to rotate about an axis of rotation 3′. The axis of rotation 3′ can be arranged for example in a central position of the inlet 10, i.e. aligned with the inlet 10. As explained above, it is assumed that the rotor only performs small translational movements, so that with the aim of providing a simpler and more understandable description, the axis of rotation can be regarded as fixed with respect to the pump chamber.

The axis of rotation 3′ can also represent a kind of axis of symmetry for the pump 1, whose components shown in FIG. 1A are arranged substantially symmetrically thereto. The pump 1 and also the rotor 3 can have an essentially radially symmetrical form and arrangement with respect to the axis of rotation 3′. Essentially means that the symmetry can also be deviated from locally.

The axis of rotation 3′ defines an axial direction 21, which can be specified as axial axis 21 in a coordinate system. A radial direction 22 can be defined perpendicular to the axial direction 21 and entered in the coordinate system as the radial axis 22. A direction of rotation with an axis of rotation perpendicular to the axial axis 21 can be specified as a tilting direction 23.

As shown in FIG. 1A, a fluid flow 13, also a flow of fluid, for example blood, can be guided initially away from the inlet 10 through the fluid connection 12 in the axial direction 21. Furthermore, the fluid flow 13 can be guided to the outlet 9, for example in the radial direction 22. The fluid can finally leave the pump chamber 2 via the outlet 9. It is possible that the fluid flow 13 at the outlet 9 has essentially no component, i.e. no component of a fluid direction, in the axial direction 21.

In addition to the fluid flow 13, which represents a main flow of fluid through the pump 1, which is indicated by large arrows in FIG. 1A, a secondary fluid flow 13′, for example a secondary blood flow 13′, may be provided, wherein less fluid flows in the secondary fluid flow 13′ than in the fluid flow 13, which is symbolized by small arrows in FIG. 1A. The secondary fluid flow 13′ can be guided for example between the rotor 3 and wall 2′, for example between a first rotor magnet 4 and the wall 2′, in this case for example in the axial direction 21 to the inlet 10.

Furthermore, a tertiary fluid flow 13″ can be provided, wherein less fluid flows in the tertiary fluid flow 13″ than in the fluid flow 13, which is symbolized by small arrows in FIG. 1A. The tertiary fluid flow 13″ can be guided for example between the rotor 3 and wall 2′, for example between a motor magnet 11 and the wall 2′, in this case for example in the radial direction 22 to the axis of rotation 3′.

The secondary fluid flow 13′ and tertiary fluid flow 13″ can each have a different flow direction and can help to fill a space between the rotor 3 and wall 2′ with fluid and keep the fluid there in motion. This can be used for example to reduce the risk of clumping of the fluid or, for example in the case of blood, reduce the risk of thrombosis.

Furthermore, FIG. 1A also shows an impeller 40. The impeller 40 can comprise for example blades and/or vanes, which are configured to drive the fluid flow 13, in particular on rotation of the impeller 40. The secondary fluid flow 13′ and the tertiary fluid flow 13″ can also be driven in this way respectively. The impeller 40 is coupled rigidly to the rotor 3 and rotates with the rotor 3 on rotation of the rotor 3. The impeller 40 has a substantially disc-like form in FIG. 1A. This means that its expansion in the axial direction 21 is smaller than in the radial direction 22, wherein an imaginary envelope (not shown) of the impeller 40 is substantially disc-like, i.e. is flat. A semi-axial impeller arrangement is also possible, which includes an axial portion and the flow direction is not abruptly changed from substantially axial to substantially non-axial.

The motor magnet 11 is arranged adjacent to the impeller 40. The motor magnet 11 can include a plurality of motor magnets. The motor magnet 11 can be a permanent magnet, for example a rare-earth magnet, e.g. NdFeB or SmCo. Furthermore, a magnetization direction of the motor magnet 11 can extend in the axial direction 21. In the case of a plurality of motor magnets 11 it is possible for neighboring motor magnets 11 to have a different direction of magnetization in each case. Other magnetization directions, for example a tangential or a radial magnetization direction, are also conceivable. It is also conceivable that the motor magnets are located in the vanes of the impeller.

The motor magnet 11 is shown opposite the motor stator 11′. The motor stator 11′ can comprise one or more motor coils (not shown). Furthermore, the motor stator 11′ is arranged at the wall 2′, for example integrated into the wall 2′ or, starting from the pump chamber 2, positioned behind the wall 2′. The motor stator 11′ is configured to generate a magnetic field with the motor coils, through which an electric current can flow, which can for example have a component in the axial direction 21, and which is further configured to interact with the magnetic field of the motor magnet 11 in order to rotationally drive the impeller 40. In such a case, the impeller 40 can rotate about the axis of rotation 3′, for example together with the rotor 3.

FIG. 1A also shows the first rotor magnet 4. The first rotor magnet 4 can be a permanent magnet for example, and it can be arranged in a ring at a distance around the axis of rotation 3′ in or also at the rotor 3. The magnetization direction of the first rotor magnet 4 can be provided for example in the radial 22 or in the axial direction 21. It can also be divided into one or more sections, for example in the axial direction 21, each with its own magnetization. The magnetization direction of the various sections can differ from one another, for example in opposite directions. In principle also other arrangements are conceivable, such as an arrangement as a ring-shaped Halbach array, in which the magnetization direction of individual sections of the Halbach array can change in relation to each other.

A first chamber magnet 4′ is arranged at the wall 2′ at a radial distance from the first rotor magnet 4 and opposite this. The first chamber magnet 4′ is arranged in the vicinity of a control coil 6. The first chamber magnet 4′ can also be arranged in a ring at a distance about the axis of rotation 3′. The magnetization direction of the first chamber magnet 4′ can be provided for example in the radial direction 22 or in the axial direction 21. It can also be divided into one or more sections, for example in the axial direction 21, each with its own magnetization. The magnetization direction of the various sections can differ from one another, for example in opposite directions. In principle also other arrangements are conceivable, such as an arrangement as a ring-shaped Halbach array, in which the magnetization direction of individual sections of the Halbach array can change in relation to each other. The chamber magnet can also be spaced axially from the rotor magnet which creates an axial force on this bearing. The force can for example quasi-statically counteract the force of attraction of the flow-directing elements 7 around the control coil 6 or reinforce this force of attraction.

The magnetization of the first rotor magnet 4 and the first chamber magnet 4′ is performed in this example so that they both repel one another.

In FIG. 1A the first rotor magnet 4 and the first chamber magnet 4′ together form a first magnetic bearing 81. The first magnetic bearing 81 in the case shown in FIG. 1A is a permanent magnetic bearing or a passive magnetic bearing.

FIG. 1A also shows a second rotor magnet 5. The second rotor magnet 5 can be a permanent magnet for example and it can be arranged in a ring around an inner circumference 41 of the impeller 40 at a distance around the axis of rotation 3′. The magnetization direction of the second rotor magnet 5 can be provided for example in the radial direction 22 or in the axial direction 21. It can also be divided into one or more sections, for example in the axial direction 21, each with its own magnetization. The magnetization direction of the various sections can differ from one another, for example in opposite directions. In principle also other arrangements are conceivable, such as an arrangement as a ring-shaped Halbach array, in which the magnetization direction of individual sections of the Halbach array can change in relation to each other.

A second chamber magnet 5′ is arranged opposite the second rotor magnet 5 at the wall 2′. The second chamber magnet 5′ can also be ring-shaped or cylindrical for example. It is possible to arrange the second chamber magnet 5′ so that the axis of rotation 3′ runs through the second chamber magnet 5′. The axis of rotation 3′ can be aligned with a cylindrical axis of the second chamber magnet 5′, in particular if the second chamber magnet 5′ has cylindrical or a ring-shaped form, for example in the form of a hollow cylinder. The magnetization direction of the second chamber magnet 5′ can be provided for example in the radial direction 22 or the axial direction 21. It can also be divided into one or more sections, for example in the axial direction 21, each with its own magnetization. The magnetization direction of the various sections can differ from one another, for example in opposite directions. In principle also other arrangements are conceivable, such as an arrangement as a ring-shaped Halbach array, in which the magnetization direction of individual sections of the Halbach array can change in relation to each other.

The magnetization of the second rotor magnet 5 and the second chamber magnet 5′ is performed in this example so that they both repel one another.

First rotor magnet 4, second rotor magnet 5, first chamber magnet 4′ and second chamber magnet 5′ can be made with neodymium magnets for example.

In this example, the second rotor magnet 5 and the second chamber magnet 5′ together form a second magnetic bearing 82. In the case shown in FIG. 1A, the second magnetic bearing is a permanent magnetic bearing or a passive magnetic bearing.

In a first variant, the magnetization of the chamber magnet 4′ points substantially in the direction of the rotor magnet 4, whereas the magnetization of the rotor magnet 4 points in the direction of the chamber magnet 4′. In this case, the first magnetic bearing is both radially and axially repelling, whereby the first magnetic bearing is axially unstable and radially stable. The second magnetic bearing can be magnetized in the same way: The magnetization of the second chamber magnet 5′ points radially outwards, whereas the magnetization of the second rotor magnet points inwards so that the magnetizations of the two magnets are directed substantially towards one another.

In a second variant, the magnetization of the first chamber magnet and the magnetization of the first rotor magnet are directed upstream (or downstream) essentially parallel to one another. In this case, the first magnetic bearing is both radially and axially repelling, whereby the first magnetic bearing is axially unstable and radially stable. Likewise, the second magnetic bearing can also be magnetized such that the magnetizations of the second rotor and the second chamber magnet are parallel to one another and point in essentially the same direction as the magnetization of the first magnetic bearing.

The two aforementioned variants can also be combined with one another, i.e. the first magnetic bearing is configured according to the first variant, the second magnetic bearing according to the second variant or vice versa.

In a third variant, both the first (or second) chamber magnet and the first (or second) rotor magnet can be configured as a squeeze-field magnet, i.e. the magnetization of the upstream half of the respective magnet point downstream and the downstream half points upstream. To increase the rigidity of the magnetic bearing a plurality of squeeze-field pairs can be formed in succession in axial direction. In this case, the first (or second) magnetic bearing is both radially and axially repelling, wherein the first (or second) magnetic bearing is axially unstable and radially stable.

FIG. 1A also shows the control coil 6. In the representation shown, the control coil 6 is arranged completely between the inlet 10 and the rotor 3, as viewed in the axial direction 21. One turn of a winding of the control coil 6 is guided around the fluid connection 12 and thus around the pump chamber 2 at a distance from the axis of rotation 3′, i.e. around the axial direction 21. A magnetic field that can be generated by the control coil 6 can have a component in the axial direction 21. The magnetic field of the control coil 6 is also configured to interact with a magnetic field of the first rotor magnet 4 and to exert an axial force on the rotor in the axial direction 21. The magnetic field of the control coil 6 can be generated by a control current. The axial force on the rotor 3 can be controlled by the control current for example.

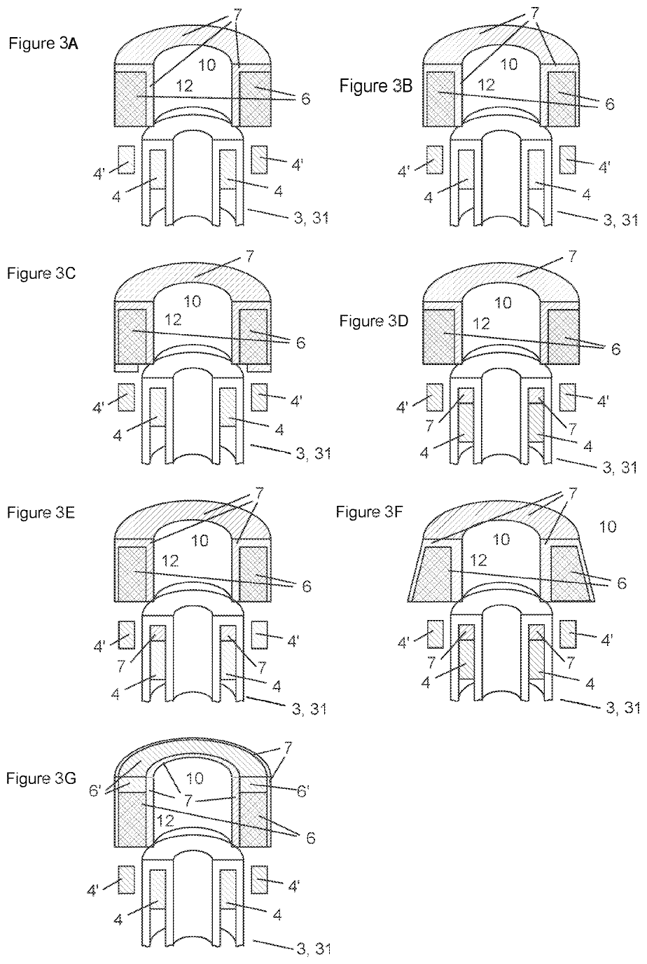

A magnetic material 7 is also shown adjacent to the control coil 6 in FIG. 1A. The magnetic material 7 is used for guiding the magnetic field generated by the control coil and guides an associated magnetic flow, so that a greater axial force can be generated than without the magnetic material 7. Further details of the arrangement of the magnetic material 7 will be considered in more detail in the discussion of FIGS. 3A-3G. The magnetic material 7 can be integrated into the wall 2′ and/or form a part of the wall 2′ and/or also be arranged at the wall 2′.

In an alternative variant to the positioning of the control coil shown in FIG. 1A, the control coil can have one part which is arranged upstream of the first chamber magnet and one further part that is arranged downstream of the first chamber magnet. Alternatively, two separate control coils can also be arranged in this way. Here it is advantageous, if the downstream part or the second control coil is arranged upstream of the second chamber magnet or in the vicinity of the first chamber magnet. The control coil can generate a magnetic field which is suitable for example for generating a magnetic field in a first magnetic bearing made up of squeeze-field magnets, which improves or enables axial positioning of the rotor.

In addition, FIG. 1A also shows a control unit 60. The control unit 60 can provide a control current for the control coil 6. The control current can be used for example to control a strength of the magnetic field of the control coil 6. This can ultimately be used to control the axial force on the rotor 3, which can be generated by the interaction of the magnetic field generated by the control coil 6 and the magnetic field of the first rotor magnet 4. The control current can thus be provided in such a way that the rotor 3 is held in a position spaced axially from the wall 2′ of the pump chamber 2, for example in that the control current is large enough in each case to generate an axial force which is necessary to hold it in the axially spaced position. Forces on the rotor can be forces for example which are caused by the effect of the magnetic fields of the first rotor magnet 4, the second rotor magnet 5, the first chamber magnet 4′ and the second chamber magnet 5′ and/or also external forces on the rotor 3, for example based on the fluid flow 13, gravitation and/or an accelerated movement of the pump 1.

The control unit 60 can be configured for example as a microcontroller, a digital signal processor, an FPGA, or ASIC or also a combination thereof and can also contain other electronic components. These may also include sensors for example, such as for example a distance or also position sensor, for example for determining a position of the rotor 3, for example in the axial direction 21. Other possible sensors include for example a magnetic field sensor, a speed sensor, a pressure sensor, a fluid flow sensor and the like.

The control unit 60 can also be used to control the motor stator 11′, for example by supplying the motor coil of the motor stator 11′ with an electric current from the control unit 60, for example a time-varying electric current. For example, this electric current can be such that a magnetic field suitable for a rotary drive of the pump 1 is generated, wherein the magnetic field of the motor coil can interact with the magnetic field of the motor magnet for this purpose. However, the current provided by the control unit 60 for the motor stator 11′ can also be configured to generate an axial force in the axial direction 21 on the rotor 3, also in cooperation with the motor magnet 11. For this purpose, the control unit 60 can comprise a controller which operates for example according to the principle of a field-operated control. The axial force generated by the motor stator 11′ with the motor magnet 11 can be coordinated with the generation of the axial force by the control coil 6 with the first rotor magnet 4. It is possible for example to configure the axial force generated by the motor, comprising the motor stator 11′ and motor magnet 11, as a biasing force for the axial force generated by the control coil 6. For example the biasing force can be speed-dependent.

FIG. 1B shows a cross-sectional representation of the rotor 3 from FIG. 1A. Here the rotor 3 has a first section 31 and a second section 32. For example, the first motor magnet 4 is arranged in the first section 31. In the second section 32 the impeller 40 is shown as well as the motor magnet 11 and the second rotor magnet 5. In this respect the first section 31 comprises the first magnetic bearing 81 and the second section comprises the second magnetic bearing 82 and the impeller 40. The rotor can be fully supported in the radial direction 22 by the first and the second magnetic bearing 81, 82. The first and second magnetic bearings 81, 82 are spaced apart from one another in axial direction. This means for example that a movement of the rotor 3 in the tilting direction 23 can also be at least partially suppressed.

The rotor 3 also has a recess 8 arranged around the axis of rotation 3′ (not shown in FIG. 1A for reasons of clarity). For example, the recess 8 can be radially symmetrical about the axis of rotation 3′, for example in the form of a bore. The recess 8 has the advantage that the fluid can be guided through it. Thus it is possible for example to keep the distance between the first rotor magnet 4 and the first chamber magnet 4′ as small as possible, as the fluid does not have to be guided between the first rotor magnet 4 and the first chamber magnet 4′, but can flow through the recess 8. This allows the distance-dependent magnetic forces between the first rotor magnet 4 and the first chamber magnet 4′ to be as great as possible. In addition, the magnets themselves can be kept small and thus the installation space of the pump 1 can be kept small. Viewed in radial direction, a cross-section of the recess 8 can correspond for example or also essentially correspond to a cross-section of the inlet 10 in terms of the form and area with the aim of minimizing the flow resistance for the fluid through the pump 1. Here is also possible that the cross-sections of the recess 8 and inlet 10 are aligned relative to one another, as indicated in FIG. 1A.

Both the first section 31 and the second section 32 can comprise the recess 8 and in this way guide the fluid towards the impeller 40, so that it can flow from there to the outlet 9 or can be driven there by the impeller 40.

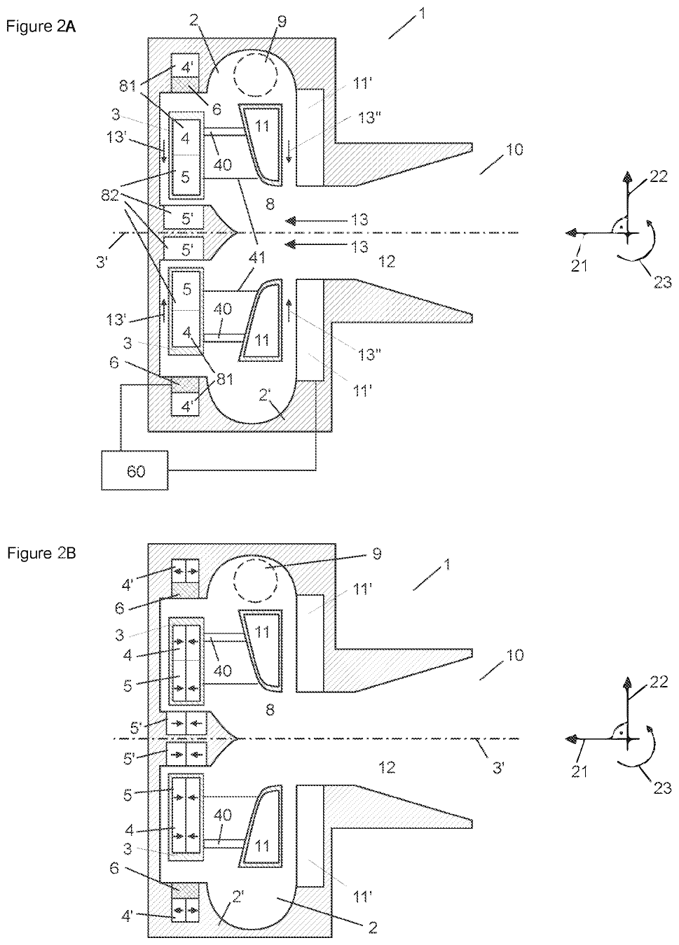

FIG. 2A shows a schematic representation of a pump 1 according to the present disclosure which has a number of similarities with the pump 1 shown in FIG. 1A, which will not be discussed again here. Rather, the differences from the pump 1 shown in FIG. 1A are not discussed here. Thus in the pump 1 shown in FIG. 2A, also in a cross-sectional view, the motor stator 11′ is arranged at the pump chamber 2 on one side of the impeller 40 facing the inlet 10. The motor magnets 11 are arranged opposite in the axial direction at the impeller 40. The motor magnets 11 and motor stator 11′ differ here from the example shown in FIG. 1A only with regard to their arrangement in the pump 1, but not with regard to their function and the further details described above.

Furthermore, in FIG. 2A the first rotor magnet 4 is arranged in the vicinity of an outer circumference of the impeller 40, in this case on a side of the impeller 40 facing away from the inlet 10. The first chamber magnet 4′ is spaced apart radially from the first rotor magnet 4 at the wall 2′ of the pump chamber 2. The first chamber magnet 4′ is adjacent to the winding of the control coil 6. The winding of the control coil 6 is arranged between the first chamber magnet 4″ and the pump chamber 2. One turn of the winding is guided around the pump chamber 2 in the axial direction 21. This enables a high efficiency of the control coil 6, wherein this refers to a high force effect that can be generated (together with the first rotor magnet 4) per unit of the electrical control coil current. As shown, in contrast to FIG. 1A, the control coil 6 is therefore not arranged in the vicinity of the inlet 10 but in the vicinity of the outlet or in the vicinity of the impeller. However, the control coil 6, together with the first rotor magnet 4, can also exert a force on the rotor 3 in the axial direction 21. Furthermore, due to the distance of the first rotor magnet 4 and the control coil 6 from the axis of rotation 3′, the force generated by the first rotor magnet 4 and the control coil 6 can reduce a movement of the rotor 3 in the tilting direction 23.

This is also the task of the first rotor magnet 4 together with the first chamber magnet 4′, whose magnetic fields interact and generate a force on the rotor. In this example this force can be a force of attraction between the first rotor magnet 4 and first chamber magnet 4′. In principle, the force generated in this way can also act on the rotor 3 in the axial direction 21 and in the tilting direction 23.

The second rotor magnet 5 and the second chamber magnet 5′ are substantially similar to the corresponding elements of FIG. 1A. In FIGS. 2A and 2B, the first rotor magnet 4 and the second rotor magnet 5 are configured as separate magnets. For example the advantage here is that the magnetization of both rotor magnets can be different according to polarization and/or strength and thus the respective forces acting in the first magnetic bearing 81 or in the second magnetic bearing 82 can be configured independently of one another.

In addition, it is also possible that the second rotor magnet 5 and the first rotor magnet 4 are combined into a single rotor magnet. This is possible for example if both have a correspondingly coordinated magnetization. This is shown in FIG. 2B for example.

In FIG. 2B, which is based on FIG. 2A, the rotor or chamber magnets 4, 4′, 5, 5′ shown each have several sections in the axial direction 21. Each of the sections shown has a magnetization, shown by arrows in the representations of the respective magnets 4, 4′, 5, 5′, in the axial direction 21, wherein in principle a magnetization in the radial direction 22 would also be conceivable in each case. However, the magnetization in the axial direction 21 facilitates the manufacture of the magnets and the assembly of the pump 1. Here in the example shown, due to the respective direction of the arrow it is clear that the first rotor magnet 4 and the second rotor magnet 5 each have identically magnetized sections in the axial direction 21, which means that the first and the second rotor magnet 4, 5 can be replaced by a single magnet, for example divided into the axial sections shown. An alternating magnetization of the first rotor magnet 4 and second rotor magnet 5 is also conceivable.

FIGS. 3A to 3G show schematic representations of the first section 31 of the rotor 3 and the control coil 6 in a cross-sectional view and in part a perspective view and thus continue the example from FIGS. 1A and 1B. Relative directions are adopted, as defined in FIGS. 1A and 1B. Details that are not shown correspond substantially to the representations in FIGS. 1A and 1B. In particular, FIGS. 3A to 3G show respectively the rotor 3, the first rotor magnet 4 and the first chamber magnet 4′, the control coil 6 and the magnetic material 7.

FIG. 3A shows a possible arrangement of the magnetically conductive material 7, and in each case at the control coil 6, wherein the magnetically conductive material 7 is placed on the side of the control coil 6 facing the inlet 10 and on the side of the control coil 6 facing the fluid connection 12, i.e. between the fluid connection 12 and control coil 6, and is also placed here adjacent to the winding of the control coil 6. Turns of a winding of the control coil are guided around the fluid connection 12. One turn of the winding is thus also guided about the axial direction 21. The side of the control coil 6 facing away from the fluid connection 12 and the side of the control coil 6 facing the rotor 3 are not provided with the magnetically conductive material 7. The magnetic field generated by the control coil 6 can be conducted in the magnetically conductive material 7, which increases the magnetic flow density in the gap to the rotor magnet 4. This means that for the same control current a greater local magnetic flow density and therefore also a greater axial force can be generated between the control coil 6 and rotor 3. This is case for example in FIG. 3A at the point where the magnetically conductive material 7 comes closest to the rotor 3 or in this case also the first rotor magnet 4. The axial force effect is greatest in this area.

The advantages of this arrangement of the magnetically conductive material 7 are that with a given control current in the gap to the rotor 3, a greater magnetic flow density can be obtained than without magnetically conductive material 7, so that the desired axial force effect between the control coil 6 and rotor 3 can be increased at the desired point. For example, as explained above, a magnetically conductive material 7 can have a high relative magnetic permeability such as a ferromagnetic material, for example iron. Typical values for the relative magnetic permeability of ferromagnetic materials can be in the range of 300 and 10000 for example, wherein for certain materials, such as for example mu-metals, amorphous metals or also nanocrystalline metals, values significantly higher than this are known, for example of up to 150000 or even up to 500000. Lower relative magnetic permeabilities are also known for certain materials, which can drop to around 4 with ferrites for example.

FIG. 3A also shows an offset between the first rotor magnet 4 and the first chamber magnet 4′ in the axial direction 21. With such an offset it is possible for example to generate an axial biasing force which is directed away from the inlet 10 for example. This can be used advantageously to reduce the amount of stationary force provided by the control coil 6 and the first rotor magnet 4 in the axial direction and for example to thus increase the efficiency of the pump 1.

FIG. 3A shows a variant of FIG. 3A, wherein here, in addition to FIG. 3A, the magnetically conductive material 7 is also placed on the side of the control coil 6 facing away from the fluid connection 12. The magnetically conductive material 7 can for example have a different material thickness in the radial direction 22 on the side of the control coil 6 facing the fluid connection 12 than on the side of the control coil 6 facing away from the fluid connection 12. For example, as shown here, (as viewed in radial direction 22) the material thickness of the magnetically conductive material 7 on the side of the control coil 6 facing the fluid connection 12 may be greater than on the side of the control coil 6 facing away from the fluid connection 12. The ratio of the material thicknesses can for example be such that a cross-sectional area of the magnetically conductive material 7 on the side of the control coil 6 facing the fluid connection is approximately equal to a cross-sectional area of the magnetically conductive material 7 on the side of the control coil facing away from the fluid connection, wherein the cross-sectional area is viewed perpendicular to the axial direction. The advantage of this is that the magnetic material is used as uniformly as possible with regard to a material-specific maximum magnetic flow density to be considered.

Based on FIG. 3B, FIG. 3C shows a further variant of the arrangement of the magnetically conductive material 7. In FIG. 3C in addition to the arrangement in FIG. 3B magnetically conductive material 7 is also arranged on the side of the control coil 6 facing the rotor 3. The magnetically conductive material 7 can be arranged such that the gap between the rotor 3 and control coil 6 is not enlarged compared to the example in FIG. 3B, for example in which only a portion of the side of the control coil 6 facing the rotor 3 is provided with magnetically conductive material 7. For example, a section of the side of the control coil 6 facing the rotor 3, which is directly opposite the rotor 3, is free of magnetically conductive material 7. With this arrangement the force effect can be further focused and it is also possible to further increase the magnetic flow, in particular in the gap between the rotor 3 and control coil 6, compared to the arrangement in FIG. 3C.

FIG. 3D shows a further arrangement of the magnetically conductive materials 7 similar to FIG. 3A. In addition, to the arrangement in FIG. 3A however there is section of magnetically conductive material 7 that is positioned in the rotor 3 on a side facing the inlet 10 and adjacent to the first rotor magnet 4. In this case, the magnetically conductive material 7 can be configured as an iron yoke for example and can further concentrate the magnetic flow.

FIG. 3E shows for example a combination of the examples from FIGS. 3B and 3D. It is clear that the above examples of the arrangement of the magnetically conductive material 7 can be combined as desired. This also applies in the same way to FIG. 3F, which is based on FIG. 3E, where the control coil 6 has the outer form of truncated cone compared to the previous examples. This shows, inter alia, that the control coil 6 can be formed with different geometries, which also includes geometries not shown, such as a cuboid geometry or also generally a cylindrical geometry, which also includes a ring-shaped geometry, with a polygonal base.

A further example, again with a substantially cylindrical geometry, is shown in FIG. 3G. The example shown in FIG. 3G is based on the example in FIG. 3B, wherein magnetically conductive material 7 is arranged on the side of the control coil 6 facing the inlet 10. Instead of this however, a biasing magnet 6′, for example a permanent magnet, is arranged there adjacent to the control coil 6. In the example of FIG. 3G the magnetically conductive material 7 adjoins the biasing magnet 6′ in the radial direction 22 on an inner and an outer circumference of the annular biasing magnet 6′ in this example. The magnetization of the biasing magnet 6′ can be performed here in the radial direction 22 for example. Configured as a permanent magnet, the biasing magnet 6′ can for example provide a magnetic flow without requiring an electrical control current through the winding of the control coil 6. Thus, for example an operating point of the control coil 6 together with the first rotor magnet 4 can be set with regard to the magnetic flow and/or the axial force.

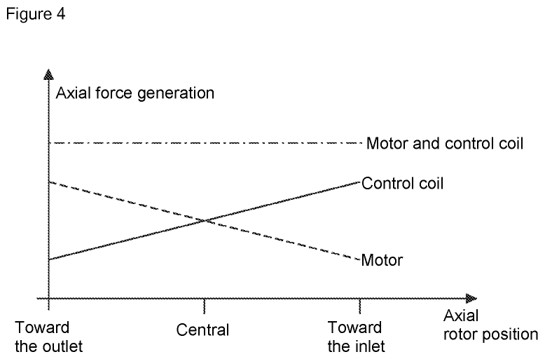

FIG. 4 shows a diagram for the option of axial force generation as a function of an axial position of the rotor 3 and relates mainly to the example in FIGS. 1A and 1B, but can be adapted in an analogous manner to the example in FIGS. 2A and 2B. The possibility is considered that both the motor which comprises the motor stator 11′ with the motor coil and the motor magnet 11, and the control coil 6, when it interacts with the first rotor magnet 4, can generate an axial force acting in the axial direction 21 on the rotor 3. The motor 11, 11′ and control coil 6 with the first rotor magnet 4 can be synchronized in their function so that the total axial force generated is substantially constant. This is symbolized by the constant horizontal dash-dot line in FIG. 4. This is advantageous, as in this way the axial position of the rotor 3 can be controlled more easily.

The solid, rising line in the diagram of FIG. 4 relates to the force generated by the control coil 6 together with the first rotor magnet 4 in the axial direction. It increases (with the same control current) the closer an axial position of the rotor 3 approaches the inlet 10.

The dashed, descending line in the diagram in FIG. 4 refers to the force generated by the motor in the axial direction. It decreases (with otherwise identical motor control) the closer an axial position of the rotor 3 approaches the inlet 10.

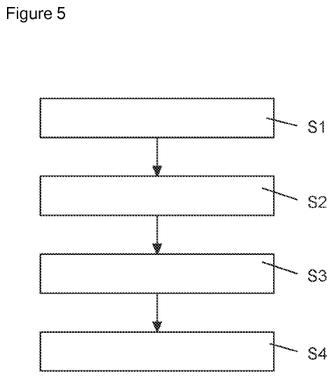

FIG. 5 shows a flow chart for a method for operating a pump 1. The method comprises the steps:
S1: Providing a motor magnetic field for driving an impeller 40 connected to a rotor 3 for driving a fluid flow 13 from an inlet 10 to an outlet 9 of a pump chamber 2;
S2: Providing, with a control coil 6 and a first rotor magnet 4 arranged at the rotor 3, a controllable axial force on the rotor 3 in the direction of an axis of rotation 3′ of the rotor 3, which defines an axial direction 21;
S3: Providing a control current for the control coil 6, to maintain the rotor 3 in a position spaced axially from a wall 2′ of the pump chamber 2, such as in a time-varying position, in which the sum of all forces on the rotor 3 except the controllable axial force in the axial direction 21 adds up to zero;
S4: Providing a force between the first rotor magnet 4 and a first chamber magnet 4′ arranged at the pump chamber 2 near a winding of the control coil 6.

The said steps can be carried out in this order but can also be carried out in a different order. Individual or several of the steps can also be performed multiple times.

As part of the method, a zero force control may be used, in which a particularly small control current is required, since the rotor 3 is held in the position which may vary over time in which the sum of all forces on the rotor 3 except the controllable axial force in the axial direction 21 adds up to zero. This enables an efficient operation of the pump 1 with minimized electrical control power.

### LIST OF REFERENCE SIGNS

1 pump
2 pump chamber
2′ wall
3 rotor
3′ axis of rotation
4 first rotor magnet
4′ first chamber magnet
5 second rotor magnet
5 second chamber magnet
6 control coil
6 biasing magnet
7 magnetically conductive material
8 recess
9 outlet
10 inlet
11′ motor stator
11 motor magnet
12 fluid connection
13 fluid flow
13′ secondary fluid flow
13″ tertiary fluid flow
21 axial direction
22 radial direction
23 tilting direction
31 first section (of the rotor)
32 second section (of the rotor)
40 impeller
41 inner circumference of the impeller
60 control unit
81 first magnetic bearing
82 second magnetic bearing

1. A pump comprising:
a pump chamber with a wall and a fluid connection for a fluid flow between an inlet and an outlet of the pump chamber;
an impeller disposed in the pump chamber and coupled to a rotor, the impeller being configured to rotate about an axis of rotation that is aligned with the inlet and defines an axial direction, and is further configured to drive the fluid flow from the inlet to the outlet;
a motor stator disposed at the pump chamber, the motor stator comprising at least one motor coil which is configured to provide a motor magnetic field to interact with a magnetic field of a motor magnet disposed on the impeller to rotationally drive the impeller;
a control coil disposed at the pump chamber, wherein one or more turns of a winding of the control coil are disposed at the wall around the pump chamber, wherein the control coil is configured to generate a magnetic field by a control current, wherein the generated magnetic field exerts a controllable axial force on the rotor in the axial direction along with a first rotor magnet arranged on the rotor;
a control unit providing the control current for the control coil, and to hold the rotor in a position spaced apart axially from the wall of the pump chamber; and
a first chamber magnet, disposed at the pump chamber, close to the winding of the control coil and opposite the first rotor magnet, wherein the first chamber magnet is configured, along with the first rotor magnet to provide a force between the first rotor magnet and the first chamber magnet to align the rotor relative to the wall of the pump chamber.

2. The pump according to claim 1, the pump further comprising:
a second magnetic bearing;
a second chamber magnet, disposed at the pump chamber where the axis of rotation meets the wall; and
a second rotor magnet disposed opposite the second chamber magnet at the rotor, wherein the second magnetic bearing is configured to provide a repelling force between the second rotor magnet and the second chamber magnet to maintain at least a portion of the rotor in a position spaced from the wall of the pump chamber in a radial direction perpendicular to the axial direction.

3. The pump according to claim 1, wherein the rotor has a recess around the axis of rotation and is configured to guide the fluid flow through the recess.

4. The pump according to claim 2, wherein the force between the first rotor magnet and the first chamber magnet comprises a force of attraction, wherein the first rotor magnet and the first chamber magnet form a first magnetic bearing that is configured to orient the rotor in a tilting direction which has an axis of rotation in the radial direction.

5. The pump according to claim 1, wherein the force between the first rotor magnet and the first chamber magnet comprises a repelling force in a radial direction which is perpendicular to the axial direction, wherein the first rotor magnet and the first chamber magnet form a first magnetic bearing that is configured to maintain the rotor in a position spaced apart from the wall of the pump chamber in the radial direction.

6. The pump according to claim 5, wherein the control coil comprises a magnetically conductive material arranged between the winding and the fluid connection or on a side of the control coil facing away from the rotor.

7. The pump according to claim 5, further comprising:
a biasing magnet disposed at the control coil and configured to bias the magnetic field of the control coil.

8. The pump according to claim 4, wherein the second rotor magnet and the second chamber magnet are disposed relative to one another so that an axial force acts on the rotor in the axial direction away from the inlet, and a quotient of a radial rigidity of the second magnetic bearing to a radial rigidity of the first magnetic bearing in the event of rotor displacement in the axial direction, the radial direction or a tilting direction, which has an axis of rotation in the radial direction, remains substantially constant, wherein the radial rigidity of the first magnetic bearing is a result of the rigidity of the control coil and first chamber magnet with the first rotor magnet.

9. The pump according to claim 4, wherein a ratio between a radial rigidity of the second magnetic bearing and a radial rigidity of the first magnetic bearing is between 4:1 and 1.5:1.

10. The pump according to claim 1, wherein the motor magnet is arranged on a side of the impeller facing away from the inlet or on a side of the impeller facing the inlet.

11. The pump according to claim 1, wherein the control unit is configured to provide a motor coil current such that the motor magnetic field together with the magnetic field of the motor magnet creates a force in the axial direction between the motor magnet and the motor stator.

12. The pump according to claim 1, wherein the fluid flow comprises blood flow.

13. The pump according to claim 1, wherein the pump being used in a cardiac support system.

14. The pump according to claim 1, wherein parts of the control coil are arranged in axial direction in front of and behind the magnetic bearing pairs and.

15. The pump according to claim 14, wherein a second control coil is coupled to the control coil, and disposed in axial direction upstream and the second control coil in axial direction downstream of the first rotor magnet and the first chamber magnet.

16. The pump according to any claim 1, wherein the force between the first rotor magnet and the first chamber magnet is a repellent force in an axial direction, such that the rotor is held in a position spaced apart from the wall of the pump chamber in the axial direction.

17. The pump according to claim 4, wherein the force between the first rotor magnet and the first chamber magnet is additionally a repelling force in an axial direction, so that the rotor is held in a position spaced apart from the wall of the pump chamber in the axial direction.

18. A method for operating a pump, the method comprising:
providing a motor magnetic field for driving an impeller connected to a rotor for driving a fluid flow from an inlet to an outlet of a pump chamber;
providing, with a control coil and a first rotor magnet arranged on the rotor, a controllable axial force to the rotor in the direction of an axis of rotation of the rotor, which defines an axial direction;
providing a control current for the control coil, to keep the rotor in a position spaced apart axially from a wall of the pump chamber, in a time-varying position, in which the sum of all forces on the rotor except the controllable axial force in the axial direction adds up to zero; and
providing a force between the first rotor magnet and a first chamber magnet arranged on the pump chamber near a winding of the control coil.

19. The method for operating a pump according to claim 18, comprising one or more of the steps:
guiding the fluid flow through a recess in the rotor about the axis of rotation of the rotor away from the inlet in the direction of the outlet;
guiding the fluid flow, so that the fluid flow is guided though the inlet substantially in the axial direction and is guided through the outlet in a direction which has substantially no axial component; or
guiding a secondary fluid flow between the first rotor magnet and the wall of the pump chamber or guiding a tertiary fluid flow between the motor magnet and the wall of the pump chamber.

20. The method for operating a pump according to claim 18, comprising:
generating a repelling force between a second rotor magnet and a second chamber magnet arranged on the pump chamber, where the axis of rotation meets a wall of the pump chamber.
